(19) United States
(12) Patent Application Publication
(10) Pub. No.: US 2013/0066228 A1
(43) Pub. Date: Mar. 14, 2013

(54) MINIMIZING MECHANICAL TRAUMA DUE TO IMPLANTATION OF A MEDICAL DEVICE

(71) Applicants: Edmond CAPCELEA, Macquarie University (AU); Paul STODDART, Hawthorn (AU); Scott WADE, Hawthorn (AU); Natalie L. JAMES, (US)

(72) Inventors: Edmond CAPCELEA, Macquarie University (AU); Paul STODDART, Hawthorn (AU); Scott WADE, Hawthorn (AU); Natalie L. JAMES, (US)

(21) Appl. No.: 13/231,957

(22) Filed: Sep. 13, 2011

(51) Int. Cl. A61B 5/00; G02B 6/34; G02B 6/42

(52) U.S. Cl. 600/559; 385/12

(57) ABSTRACT

A medical implant component including a fiber Bragg grating sensor is described. The sensor may be located so as to be at a leading end of the implant when the implant is being inserted, allowing feedback to be provided to a surgeon of the force being applied to the leading end of the implant during implantation. An interrogator for a sensor fiber Bragg grating is also described. The interrogator includes a matched fiber Bragg grating to the sensor fiber Bragg grating, which passes a proportion of the return light signal from the sensor fiber Bragg grating, the intensity of which can be related to the strain/force applied to the sensor Bragg grating.

### FIELD OF THE INVENTION

Aspects of the present invention generally relate to implantable medical devices, and more particularly, to minimizing mechanical trauma due to implantation of a medical device.

### RELATED ART

Many types of medical devices are commonly available to provide therapy to a patient. Some medical devices are temporarily or permanently implanted in the patient Implantation of a medical device is subject to the same complications as any other invasive medical procedure. Depending on the relative dimensions and flexibility of the anatomy and medical device, the configuration of the implantation path, and a variety of other factors, injury is sometimes caused by the physical contact with the device during implantation. The body wound or shock caused by such physical injury, referred to herein as mechanical trauma, may have an adverse effect on the patient, surgical procedure or medical device performance.

For example, implantation of an electrode assembly of a cochlear implant may cause mechanical trauma. A cochlear implant includes a sound processor that communicates with a stimulator that drives an array of electrode contacts disposed on the distal end on the elongate electrode assembly. In operation, the electrode contacts transmits electrical stimulation signals to the neural pathways in the cochlea. Mechanical trauma to the soft tissues of the spiral ligament and basilar membrane (which supports the spiral organ and hair cells) may damage the spiral ganglion cells, reduce residual hearing and may result in the dislocation of the electrode assembly from the scalia tympani into the scala vestibuli. This may reduce the recipient's ability to process speech after implantation and may restrict the use of more advanced speech coding strategies.

### SUMMARY

In one embodiment of the present invention, an implantable medical device is disclosed. The implantable medical device comprises: a carrier member configured for implantation into a patient, the carrier member having a patient contact region; one or more operative components disposed in the carrier member; a fiber optic sensor including a fiber Bragg grating (FBG) disposed in the patient contact region of the carrier member; and, and an optical fiber extending from the FBG.

In a second embodiment of the present invention, an interrogator for a sensor fiber Bragg grating, the interrogator is disclosed. The interrogator comprises: a light source for providing incident light to the sensor fiber Bragg grating over a forward light path; a return light path for receiving return light from the sensor fiber Bragg grating; a reference fiber Bragg grating disposed in the return light path, the reference fiber Bragg grating having a grating pattern to reflect return light received from the sensor fiber Bragg grating, whereby variations in the environment of the sensor fiber Bragg grating within an operating range of the sensor fiber Bragg grating that affect the return light, result in variations in the proportion of the return light passed by the reference fiber Bragg grating; and a light detector for receiving and detecting the return light passed by the reference fiber Bragg grating.

In a third embodiment of the present invention, a method of implanting a medical implant for providing stimulation signals to the nervous system of a recipient is disclosed. The method comprises: using a medical implant comprising a flexible carrier at a leading end of the medical implant as it is implanted in the recipient, the flexible carrier carrying a fiber optic including a fiber Bragg grating; connecting the fiber optic to an interrogator, including a light detector for detecting variations in the reflection/transmission characteristics of the fiber Bragg grating responsive to forces applied to the fiber Bragg grating through the flexible carrier and provide an output indicative of the force applied to the fiber Bragg grating; and as the medical implant is implanted in the recipient, monitoring the outputx.

### BRIEF DESCRIPTION OF THE DRAWINGS

Further aspects of the present invention and further embodiments of the aspects described in the preceding paragraphs will become apparent from the following description, given by way of example and with reference to the accompanying drawings.

FIG. 1 shows a diagrammatic representation of an electrode assembly for insertion into a cochlea with a block diagram representation of an implant unit for providing stimulation signals to the electrode assembly and an embodiment of an interrogator for a fiber Bragg grating (FBG) in the electrode assembly;

FIG. 2 shows an enlarged view of the tip of an embodiment of the electrode assembly shown in FIG. 1, illustrating the location of the fiber Bragg grating;

FIG. 3 shows a cross section through the electrode assembly shown in FIG. 1;

FIG. 4 shows an embodiment of a matched FBG interrogation scheme configuration for an interrogator of a fiber Bragg grating;

FIG. 5 shows the relative spectra of embodiments of a sensor FBG and a reference FBG for three instances of strain (a), (b) and (c) applied to the sensor FBG;

FIG. 6 shows a plot of normalized power measured after passing through an embodiment of the reference FBG as a function of the relative wavelength shift of the sensor FBG with respect to the reference FBG for full width at half maximum (FWHM) from 0.2 nm through to 0.5 nm;

FIG. 7 shows a plot of the relative sensitivity of an embodiment of the matched FBG method as a function of the relative wavelength shift of the sensor FBG with respect to the reference FBG;

FIG. 8 shows a plot of how the FWHM of the gratings affects the relative wavelength difference between the sensor and the reference FBG at which the peak sensitivity occurs and the ranges over which the sensitivity is greater than 20% of the peak sensitivity;

FIG. 9 shows experimental arrangements used to test an embodiment of a sensor FBG and the embodiment of the interrogation scheme illustrated in FIG. 4;

FIG. 10 shows an embodiment of the optical spectrum analyzer (OSA) spectra, recorded at several different applied strains, to show how the spectrum received varies with strain applied to an embodiment of a sensor FBG;

FIG. 11 shows a plot of the results of strain calibrations for the different pairs of gratings;

FIG. 12 shows a plot of relative sensitivity of the results shown in FIG. 11 to changes in applied strain;

FIG. 13 shows a plot illustrating the strains at which the peak sensitivity occurs and the strain ranges over which the sensitivity is greater than 20% of the peak sensitivity for the experimentally determined data; and

FIG. 14 shows the change in measured signal for a compression calibration, in accordance with certain embodiments of the invention.

### DETAILED DESCRIPTION

Aspects of the present invention are generally directed to reducing mechanical trauma caused by the implantation of medical devices. As noted, many types of medical devices are temporarily or permanently implanted in a patient. The following detailed description is provided with reference to one type of implantable medical device, namely, a cochlear implant. It will be appreciated, however, that aspects and embodiments of the present invention will also have application to other types of implantable medical devices that may cause mechanical trauma during or subsequent to implantation.

Surgical procedures, including the insertion of an electrode assembly of a cochlear implant, carry some risk. Of particular relevance is the mechanical trauma resulting from the physical injuries caused by the medical device. For example, due to the intricate and delicate nature of the cochlea, the physical constraints of the implant geometry, and other factors, implantation of an electrode assembly may result in mechanical trauma.

The inventors have identified that optical fiber Bragg gratings (FBGs) are useful to sense and monitor the mechanical resistance encountered by the electrode assembly tip during implantation. FBGs consist of a periodic modulation of the refractive index in the core of an optical fiber. When the wavelength of light propagating in the fiber is phase matched with the periodicity of the modulation, the light is reflected. Therefore external parameters, such as strain, temperature or pressure, which change the effective period of the refractive index modulation and/or the refractive index of the fiber, will cause a shift in the reflective wavelength. In strain monitoring applications, FBGs offer a linear response with a large ratio of range to sensitivity; are compact and relatively rugged when packaged appropriately and they are potentially relatively inexpensive.

Aside from the actual FBG, the other main component of an FBG sensor is the hardware used to determine the wavelength shift of the sensor FBG. Various interrogation schemes have been developed to monitor FBGs. Some of these FBG interrogation methodologies include bulk optic spectrometers (e.g., holographic gratings, prisms), tunable filters (e.g., acousto-optic, Fabry-Perot), edge filters, interferometric techniques, laser systems incorporating an active FBG, and the use of swept wavelength sources. Many of the commercial interrogation systems currently available use a method known as the swept wavelength source method, as it has a relatively high resolution, tuning range, and scanning speeds. In some embodiments, these previously available FBG interrogators are used. In other embodiments a matched FBG interrogation arrangement described below is used.

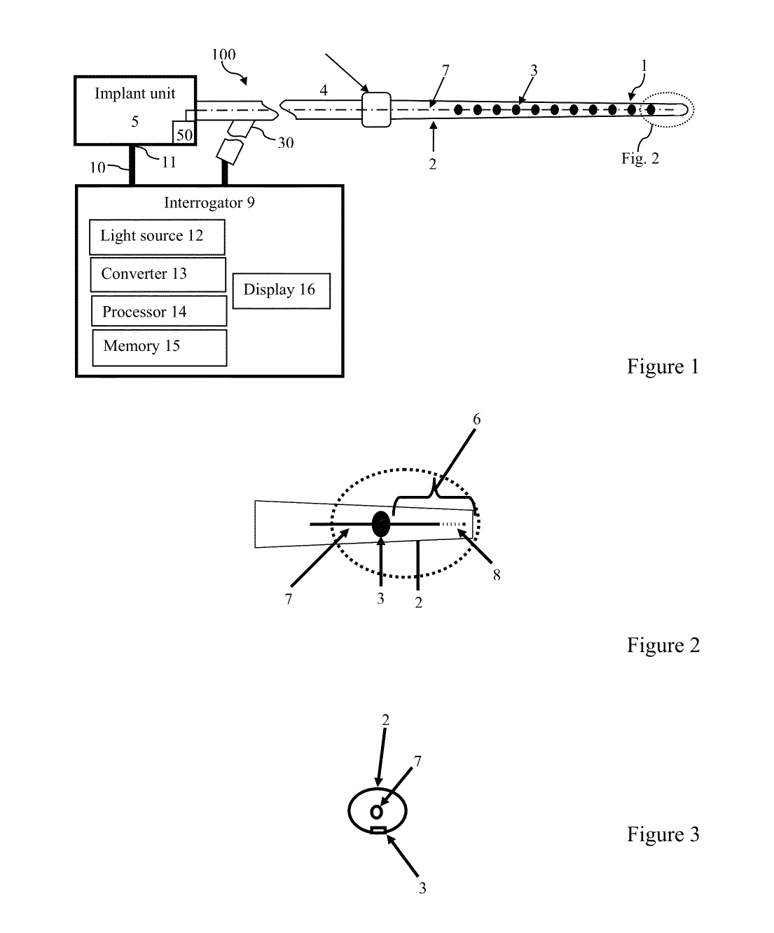

FIGS. 1 to 3 show, in diagrammatic form, an implant component 100 of a cochlear implant. FIG. 1 also shows a block diagram representation of an interrogator 9, which is described later herein.

The implant component 100 may, in use, be coupled with an external component (not shown) through coils, in which case the external component will include a sound processor and other components for the cochlear implant. Alternatively, the cochlear implant may be a totally implanted unit, in which case the implant component will include a sound processor.

Implant component 100 includes an electrode assembly 1, which includes a carrier 2, which may be silicone, and stimulation circuitry for stimulating the nervous system of the recipient, which may be in the form of an array of electrode contacts 3. Carrier member 2 includes a cochleostomy marker rib 4, to provide a guide to the surgeon as to how far electrode assembly 1 is to be inserted into the cochlea. In some embodiments, the carrier member is integrally formed. However, in other embodiments, carrier member 2 is formed in parts, joined together through welding, adhesive, over-molding or other techniques.

.Electrode assembly 1 is in electrical communication with an implant unit 5, which includes driving circuitry 50 to provide driving signals to electrode contacts 3. Driving circuitry 50 may include a coil (not shown) for receiving signals from an external sound processor and a controller and transmitter for converting the received signals into electrode stimulation signals. The signal wires connecting each electrode contact 3 to implant unit 5 have been omitted from FIGS. 1 to 3 for increased clarity of this description.

Electrode assembly 1 may be formed by placing the electrode contacts 3 and signal wires into a jig and molding the carrier 2 about the electrode contacts. The electrode assembly 1 is terminated in the implant unit 5, which includes a hermetically sealed housing. The intra-cochlea end of the electrode assembly 1 may have the general dimensions of a length (distance from the electrode assembly tip 6 to the cochleostomy marker rib 4) of about 20 mm and a diameter of between 0.4 to 0.8 mm, tapering to lesser diameters towards the electrode assembly tip 6. The electrode contacts 3 may be in a linear array. However, other suitably dimensioned and structured electrode arrays may be used with the FBG sensor, such as short electrode assemblies with an intra-cochlea length of about 8 mm. Carrier member 2, electrode contacts 3, cochleostomy marker rib 4 and implant unit 5, apart from differences explained herein, are provided in existing cochlear implants and will therefore not be described in further detail. During surgery to insert the implant component 100, the surgeon feeds electrode assembly 1 into the cochlea with the electrode assembly tip 6 leading, so that when inserted the electrode assembly 6 is the distal end relative to the location of insertion and the cochleostomy marker rib 4 is the proximal end.

An enlarged view of the electrode assembly tip 6 is shown in FIG. 2. An optical fiber 7 extends from the electrode assembly tip 6 along the electrode assembly 1. In some embodiments the optical fiber 7 is also terminated in implant unit 5 or extends through implant unit 5. When the optical fiber 7 is terminated in implant unit 5, interrogator 9 is optically connected by a suitable optical fiber 10 to a port 11 of the implant unit. When optical fiber 7 extends through implant unit 5, then optical fiber 10 is the optical fiber 7.

In other embodiments the optical fiber 7 exits carrier member 2 via a lead section 30. In such embodiments, a medical grade optical fiber connector 31 may be provided at the end of optical fiber 7. This connector can simply plug in to interrogator 9. After implantation, lead section 30 may either be cut away by the surgeon, or positioned out of the way. It will be appreciated that lead section 30 shown in FIG. 1 will be omitted if optical fiber 7 is terminated in implant unit 5.

As noted, optical fiber 7 is disposed in or on electrode assembly 1 with electrode contacts 3 and in one embodiment extends through the center of electrode assembly 1 (see FIG. 3, which shows a cross section through the electrode assembly 1 at the location of one of electrode contacts 3). The FBG, which is referenced by numeral 8 in FIG. 2, is provided at the tip region 6 of carrier member 2.

The portion of carrier member 2 forming tip region 6 has an elastic modulus that is relatively less than the elastic modulus of FBG 8 and optical fiber 7. Should tip region 6 of carrier member 2 experience strain, this relative flexibility allows for an efficient transfer of force from carrier member 2 to FBG 8. As shown in FIG. 2, tip region 6 is distal the distal-most electrode contact 3. It should be appreciated, therefore, that tip region 6 does not include electrode contacts 3 nor their associated electrical pathways since the inclusion of such elements would decrease flexibility. It should be appreciated that one or more FBG(s) 8 are disposed in the implantable medical device at location(s) that physically contact the patient in a manner that may cause mechanical trauma to the patient. In the noted example of a cochlear implant electrode assembly, such a contact region is tip region 6. In other medical devices such patient contact regions may come into physical contact with the patient during operation of the medical device rather than during implantation.

Optical fiber 7 and FBG 8 are collectively referred to herein as a fiber optic sensor. FBG 8 generates light waves having characteristics described below which are indicative of the strain imposed on tip region 6 of electrode assembly 1. The light waves are delivered to a desired location via optical fiber 7.

As noted, electrode contacts 3 are disposed in or on, or carried by, carrier member 2 of electrode assembly 1. It should be appreciated that in other implantable medical devices, other operative components may be disposed in or on the carrier that is implanted in the patient.

It should also be appreciated that carrier member 2 is flexible along its length to facilitate insertion into the cochlea, and that the carrier member of other implantable medical devices need not be flexible other than at the patient contact regions, as noted above. The optical fiber 7 may be a standard telecommunications type, photosensitive or standard low bend loss fiber. By way of example, the optical fiber 7 may have a glass cladding diameter in the range of 50-125 μm. Although optical fibers with smaller or larger diameters may be used, this range provides a balance between sensitivity (the smaller diameter fibers are more sensitive to the applied force) and flexibility (smaller fibers are more flexible and larger diameter fibers may be too stiff for a cochlear implant) and difficulty of handling during manufacture (small diameter fibers are generally more difficult to handle). In other embodiments, optical fiber 7 may be a high birefringence (Hi-Bi) fiber, which may allow for temperature correction if this is required for a particular implementation or application, or photonic crystal fiber/holey fiber/Bragg fiber, which may provide comparatively low bend losses, or polymer optical fiber, which may provide ease of compatibility with the electrode assembly.

In some embodiments, FBG 8 may be of ‘standard’ form, for example having a spectral width of <1 nm, and a reflectance of around 10 dB (i.e. ˜90%). FBG 8 may be formed using 244 nm light from a continuous-wave (CW) frequency-doubled argon-ion laser and a scanned beam phase mask method. Optical fiber 7 may be H₂ loaded prior to writing to increase photosensitivity. Other methods are known to fabricate FBGs, which may also be used to form a sensor in certain embodiments of the present invention. The grating dimensions of FBG 8 affect the range and sensitivity characteristics of the FBG sensor. In addition, the length of the grating affects the ability of the sensor to measure localized signals. Some of the FBG writing characteristics, such as reflectivity and FWHM, will also affect the sensing signal strength which may affect the sensor sensitivity and noise. The design of FBG 8 needs to take all of these into account. For the application of a cochlear implant, FBG 8 may be approximately 0.3 mm in length. Alternatively, FBG 8 may be between approximately 0.3 and 1.0 mm in length.

In other embodiments, saturated gratings, chirped gratings or phase shift gratings may be used. Some control over the sensor range and sensitivity may be achieved through appropriate selection of the FBG.

Interrogator 9 includes a light source 12, an opto-electrical converter 13, a processor 14, memory 15 and a display 16 and/or one more alternative feedback devices such as a speaker or other device for generating sound so as to provide audible feedback, or LEDs for providing alternative or additional visual feedback. Light source 12 generates light for transmission to FBG 8 via optical fiber 10, implant unit 5 and fiber optic 7. Opto-electrical converter 12 receives light reflected back from the FBG, along the same light path. Processor 14 compares the properties of the received light, for example its wavelength and/or its intensity with a measurement standard stored in memory 15. The measurement standard may, for example, be a look-up table, a threshold value or an algorithm storing a mathematical relationship between wavelength and/or intensity against a measure of the strain (or force) applied to the FBG.

As noted, there are a range of devices known for interrogating a FBG and resolving the wavelength of reflected signals, enabling determination of a measure of the force applied to the FBG. These devices may be used with the implant component described above. An interrogator using an alternative ‘matched’ FBG interrogation scheme is described below.

The matched FBG interrogation scheme uses a second wavelength-matching FBG in conjunction with sensor FBG 8 to monitor wavelength shifts that are due to the measurand of interest (e.g. strain or force in the longitudinal direction and temperature). The matched FBG has a relatively simple configuration in comparison with many of the other techniques, therefore offering potential cost benefits. In addition, this method is robust (with no moving parts), compact, can be designed for low power consumption, and is lightweight.

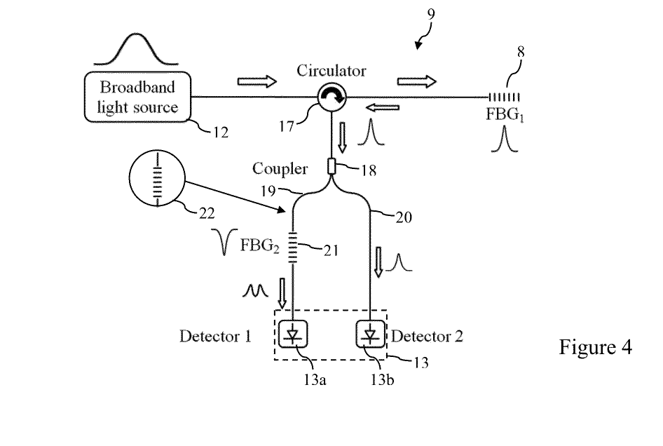

An example of an interrogator 9 including a matched FBG interrogation scheme configuration is shown in FIG. 4. Like reference numerals have been used for like components in FIGS. 1 and 4 and not all components of the interrogator are shown in FIG. 4. Interrogator 9 includes a light source 12 (for example, a broadband light source), a circulator 17 for directing the propagating and return light, a coupler 18 for splitting the return light into a first branch 19 and a second branch 20, a reference FBG 21 located along the first branch 19 and a light detector 13, including opto-electric converters 13a and 13b connected to each of the first and second branches 19, 20 respectively. Light detector 13 includes additional circuitry (not shown), such as amplifiers for opto-electric converters 13a, 13b. In other embodiments, Opto-electric converters 13a and 13b may be separate detectors, rather than parts of a single light detector. FIG. 4 also shows FBG 8, which is the sensor FBG and as such is part of the implant component, not a part of interrogator 9.

Fluctuations in the intensity of the light source or transmitted light can be compensated using coupler 18 and opto-electric converter 13b. If the intensity of the light source or transmitted light changes, the signal measured by detector 13a will also vary accordingly. Detector 13b receives a fraction of the light reflected from sensor FBG 8 and will be proportional to light source or transmitted light changes that affect detector 13a. Variations in the output of detector 13b can therefore be used to correct for non-measurand induced intensity changes at detector 13a. Where such fluctuations do not require compensation, then the coupler 18, second branch 20 and opto-electric converter 13b may be omitted. Placing reference FBG 21 in close proximity to sensor FBG 8, or varying the temperature of FBG 21 with changes in temperature of FBG 8, allows for temperature correction when using the system for strain measurements.

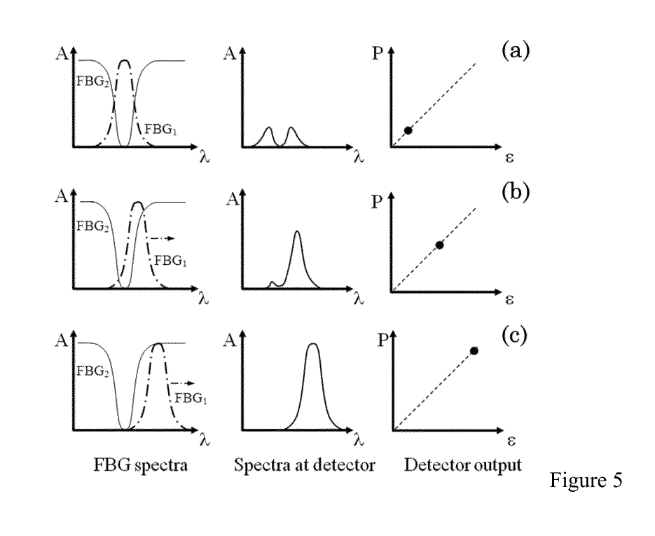

For this arrangement the spectra of the sensor and reference gratings were chosen so that they overlap as shown in FIG. 5. FIG. 5 shows the relative spectra of FBG 8 (also referenced FBG₁ in FIGS. 4 and 5) and the reference FBG 21 (also referenced FBG₂ in FIGS. 4 and 5). The spectra measured by interrogator 9 (at detector 13a) and the power measured are shown for three instances (a), (b) and (c) wherein the strain applied to FBG 8 increases from (a) to (c).

Changes in the wavelength of FBG 8 that are due to applied strain, for example, alter the extent of overlap of the two gratings. This results in a change in the magnitude of the transmitted signal to light detector 13a. Light detector 13a provides an output (see right-most graphs of FIG. 5) that indicates the level of strain applied. The output may be a numerical value showing the sensor's estimated force being applied to sensor FBG 8, a graphical display indicating the level of force being applied (e.g. a bar, the extent of which that is lit being dependent on the force applied and perhaps changing colour from green for acceptable force through to red when the force is at a level that can damage the cochlea) an audible output, or a combination of one or more of a numerical, graphical and audible output. The surgeon may use the output as an additional guide to their surgery, so that for example, the surgeon can continue inserting the electrode assembly into the cochlear despite feeling some resistance if the sensor shows that this is still at an acceptable level, or cease inserting the electrode assembly and repositioning the electrode assembly if the output indicates that the force is getting to high.

Model Assuming Gaussian Distribution for FBG Spectra. If a Gaussian distribution spectrum is assumed for both sensor FBG 8 and reference FBG 21, then the normalized spectral distribution for FBG 8, P₁(λ), is given by

$${P}_{1} ( λ ) = {A}_{1} exp ( - \frac{{( λ - {λ}_{1} )}^{2}}{2 {σ}_{1}^{2}} ) , \qquad ( 1 )$$

where A₁ is the maximum reflectance at λ₁, which is the central wavelength of FBG 8. The spectral width (FWHM) of FBG 8, Δλ₁, is related to σ₁ by

Δλ₁=σ₁2\sqrt{2 In 2} (2)

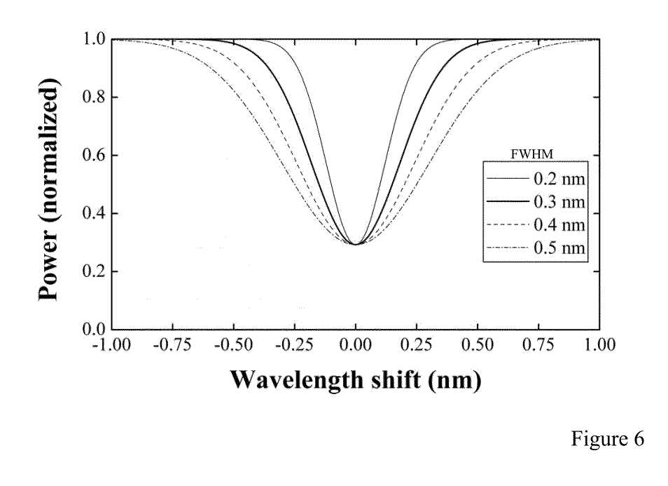

Likewise, the transmission spectrum of reference FBG 21 is given by

$${P}_{2} ( λ ) = 1 - {A}_{2} exp ( - \frac{{( λ - {λ}_{2} )}^{2}}{2 {σ}_{2}^{2}} ) , \qquad ( 3 )$$

where subscript 2 is used to denote the aforementioned characteristics for reference FBG 21. If a broadband light source is used, which effectively has a constant intensity over the wavelength range of interest, the total power measured by detector 13a is given by

$$\begin{matrix}{P}_{d} ( λ ) = {\int }_{- ∞}^{∞} {P}_{1} ( λ ) {p}_{2} ( λ ) d λ \\ = {A}_{1} {σ}_{1} \sqrt{2 π} ( 1 - \frac{{A}_{2} {σ}_{2}}{{( {σ}_{2}^{2} + {σ}_{1}^{2} )}^{1 / 2}} \times exp ( - \frac{{( {λ}_{Δ} )}^{2}}{2 ( {σ}_{2}^{2} + {σ}_{1}^{2} )} ) )\end{matrix} \qquad ( 4 )$$

where λ_{Δ}=λ₂−λ₁. FIG. 6 shows a plot of Equation (4) as a function of the relative wavelength shift of FBG 8 with respect to reference FBG 21 (i.e., for cases in which the full width at half maximum (FWHM) of both gratings is set to be equal), for a FWHM from 0.2 nm through to 0.5 nm. The maximum reflectivity of the two gratings has been assumed equal to 1 in this analysis, and the initial central wavelengths of the two gratings are assumed to be the same. FIG. 3 shows that the detected power varies more slowly as a function of wavelength shift for gratings with a larger FWHM but can potentially allow measurements over a broader range of wavelength shifts.

The relative change in measured power as a function of wavelength mismatch between the sensor and the reference FBG is given by the relative sensitivity S, which is

$$S ( {λ}_{Δ} ) = \frac{1}{{p}_{d}} \frac{d {P}_{d}}{d {λ}_{Δ}} \qquad ( 5 )$$

The sensitivity has been normalized relative to the measured power for ease of comparison. Substituting the expression for P_{d} given by Equation (4) into Equation (5) gives

$$S ( {λ}_{Δ} ) = \frac{{A}_{2} {λ}_{Δ} {σ}_{2}}{( {σ}_{2}^{2} + {σ}_{1}^{2} ) ( {A}_{2} {σ}_{2} - {( {σ}_{1}^{2} + {σ}_{2}^{2} )}^{1 / 2} exp ( \frac{{λ}_{Δ}^{2}}{2 ( {σ}_{2}^{2} + {σ}_{1}^{2} )} ) )} \qquad ( 6 )$$

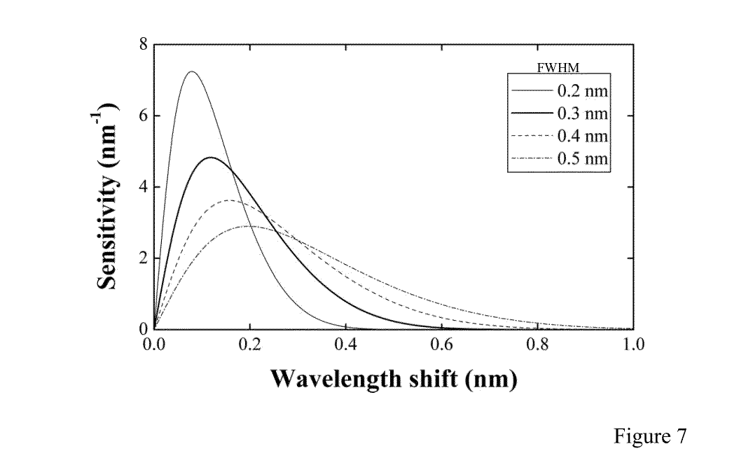

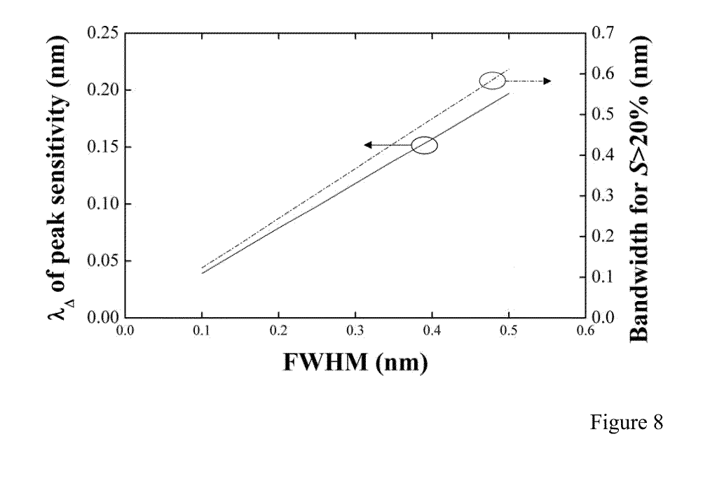

The relative sensitivity of the matched FBG method is shown in FIG. 7. For the sake of simplicity and to allow easier comparison, data have only been plotted for positive wavelength shifts of the sensor FBG relative to the reference FBG. Negative wavelength shifts result in a mirror image with inverted sign. The data in FIG. 7 show when the FWHM of the two FBGs is assumed to be the same. FIG. 7 shows that the wavelength difference between the FBG 8 and the reference FBG 21 at which the peak sensitivity occurs and the range of wavelength shifts for which the scheme is sensitive depends on the FWHM of the gratings used. FIG. 8 illustrates how the FWHM of the gratings affects the relative wavelength difference between the sensor and the reference FBG at which the peak sensitivity occurs. An indication of the effect of the grating FWHM on the range of wavelength shift that can be measured is also shown in FIG. 8 for the case when the sensitivity is greater than 20% of the maximum sensitivity measured. Both parameters are useful when designing a sensing scheme for a particular application. In particular, the sensitivity helps to determine the measurement accuracy. If all else is equal the higher the sensitivity the greater the accuracy of the measurement. The sensitivity is not constant over the possible measurement range of the system (i.e. the span of forces, or strains over which the system can measure); with this system a higher average sensitivity comes at the expense of a smaller measurement range, while a wider measurement range will give a lower average sensitivity. By altering the FWHM of the FBGs used, the system can be tailored to suit appropriate measurement ranges and sensitivities for a particular application.

An increase in the sensitivity to strain applied to FBG 8 can be achieved through the use of an additional FBG 22 with the center wavelength slightly offset from the reference grating. The additional FBG 22 blocks the unwanted light at wavelengths not relevant to the application, e.g., the peak shown on the lower wavelength side in FIG. 5(a). Consequently this arrangement reduces the detected power when the gratings are matched but has no effect on the power level for fully unmatched gratings. This increases the range of power measured and hence the overall sensitivity and also helps to prevent negative wavelength shifts being incorrectly interpreted as positive shifts and vice versa.

### Experimental Arrangement

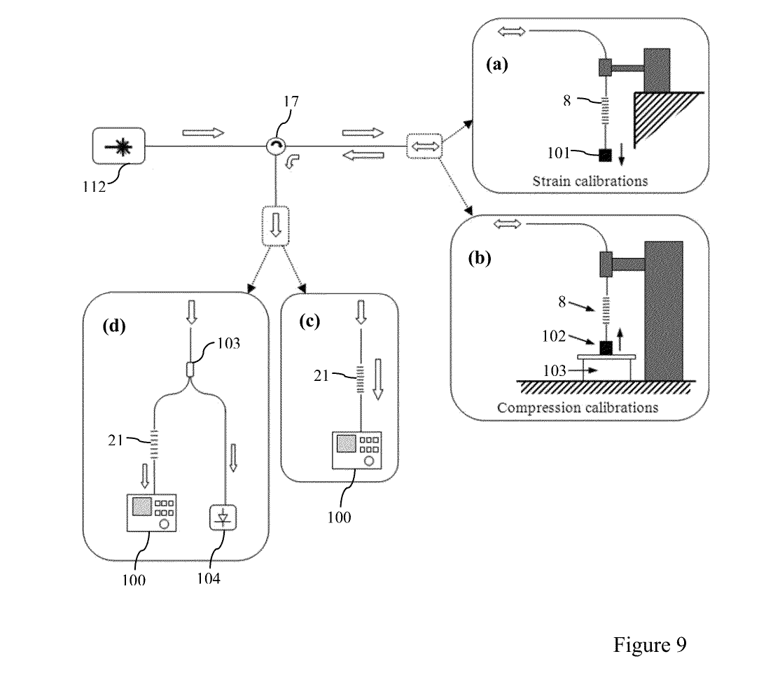

Using the arrangement shown in FIG. 4, a broadband light source of spectral width >75 nm at 3 dB of peak output, output power ˜5 mW was used to illuminate FBG 8 by means of a three-port circulator 17 with insertion losses <0.5 dB, isolation >65 dB. In each of the tests the spectra of the FBG 8 transmitted via the reference FBG 21 were measured with an optical spectrum analyzer 100 of 0.01 nm resolution, normal sweep mode, eight averages. The magnitude of the transmitted power was obtained by integrating individual spectra. FIG. 9 shows the testing arrangements.

The FBGs used were written in H₂ pre-sensitized standard telecommunications fiber ( 9/125 μm core-cladding diameter) using 244 nm radiation from a frequency-doubled argon-ion laser with the scanning phase mask technique. The center wavelengths of the gratings were in the 1550 nm region. Details of the gratings we used are provided in Table 1. In Table 1 the FBG ID column shows the FBG 8 and reference FBG 21 pair as having the same number, with FBG 8 ending with ‘s’ and the reference FBG 21 ending with ‘r’.

TABLE 1
Details of Fiber Bragg Gratings Used (λ_{c}, Center
Wavelength; R, Reflectance)

| FBG ID | λ_{c} (nm) | FWHM (nm) | R (dB) | Length (mm) |
|---|---|---|---|---|
| Fbg1s | 1550.55 | 0.26 | −12.5 | 5.0 |
| Fbg1r | 1550.50 | 0.26 | −13.0 | 5.0 |
| Fbg2s | 1550.57 | 0.31 | −17.5 | 5.0 |
| Fbg2r | 1550.56 | 0.31 | −17.7 | 5.0 |
| Fbg3s | 1549.77 | 0.35 | −23.8 | 3.0 |
| Fbg3r | 1549.78 | 0.39 | −9.6 | 3.0 |
| Fbg4s | 1550.84 | 0.50 | −12.4 | 2.0 |
| Fbg4r | 1550.76 | 0.49 | −12.2 | 2.0 |
| Fbg5s | 1549.83 | 0.54 | −8.5 | 1.4 |
| Fbg5r | 1549.85 | 0.54 | −8.3 | 1.4 |

The FBG spectral characteristics were measured at room temperature with zero applied strain, using a swept wavelength system with 3 pm resolution.

Strain calibrations were carried out by fixing one end of FBG 8 and attaching a known mass 101 to the optical fiber on the other side of the grating [FIG. 9(a)]. Applied strain ε can be calculated using

$$ɛ = \frac{mg / A}{Y} , \qquad equation ( 7 )$$

where m is the applied mass, g is the gravitational constant (9.81 m/s), A is the cross-sectional area of the fiber, and Y is Young's modulus for the optical fiber (approximately 72.5 GPa for fused silica). The elastic modulus for the silicone carrier is much smaller at approximately 0.45 MPa and so the forces applied to the tip are efficiently transferred to the grating. The optical fibers used could be either polymer based (elastic modulus of the order of 1 to 4 GPa) or silica based (elastic modulus of about 60-80 GPa). An elastic modulus in the range of 1 to 100 GPa may be suitable for testing and commercial implementation. The carrier (whether silicone or otherwise) may have an elastic modulus up to about 100 MPa.

To account for any temperature drift, several cycles (typically five) of increasing strain levels were recorded during each test and the averages reported. The variation in power measured at individual strains over the five cycles was typically of the order of 3%; as the resultant error bars would be relatively minor, they have not been plotted.

The arrangement used for compression measurements involved cutting the optical fiber close to the location of the sensor FBG 8. This fiber end was then placed in contact with a piezotranslator 102 of 15 μm travel. The piezotranslator 102 was used to apply a force to the tip of this fiber, with the fiber at the other end of the grating fixed [FIG. 9(b)]. By placing the piezotranslator 102 on top of a high resolution mass balance 103 the level of fiber compression can be calculated using a method similar to that in Equation (5).

The effect of intensity fluctuations on the system measurements was assessed by introducing a 360° bend in the optical fiber at a location between the circulator 17 and the sensor FBG 8. Reductions in the diameter of this bend caused a controllable drop in the power of light transmitted to the FBG 8 from a broadband light source 112 and also the light reflected by the grating back to the circulator 17. The arrangement used for this test incorporated a 1×2 coupler 103 of 98:2% split ratio connected to the output arm of the circulator 17 [as shown in FIG. 9(e)]. The 98% output port of the coupler was connected to the OSA 100 and the 2% port was connected to an optical powermeter 104. Although a 50:50 split ratio would normally be used, the 98:2 coupler provided an appropriate power balance between the dispersive OSA measurement and the integrated powermeter measurement.

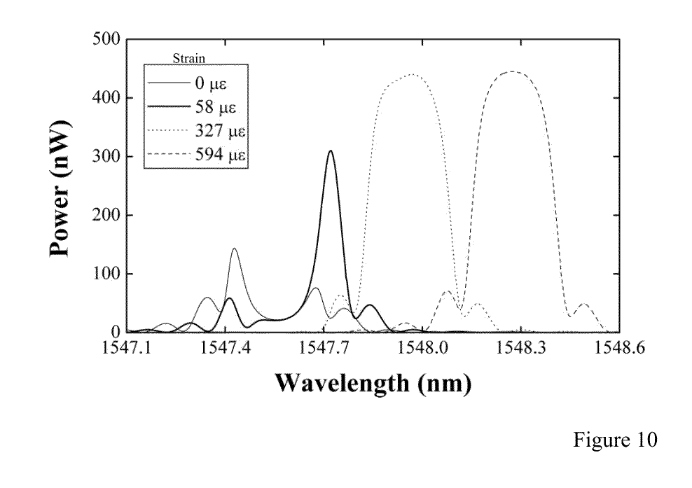

The effect of the spectral characteristics of the FBGs was investigated with five pairs of gratings with the FWHM ranging from 0.26 to 0.54 nm (as listed in Table 1). Both strain and compression calibrations were carried out. FIG. 10 gives examples of OSA spectra, recorded at several different applied strains, to show how the spectrum received by the detector 13a varies with applied strain. The spectrum shifts to longer wavelengths and the intensity increases as the applied strain increases.

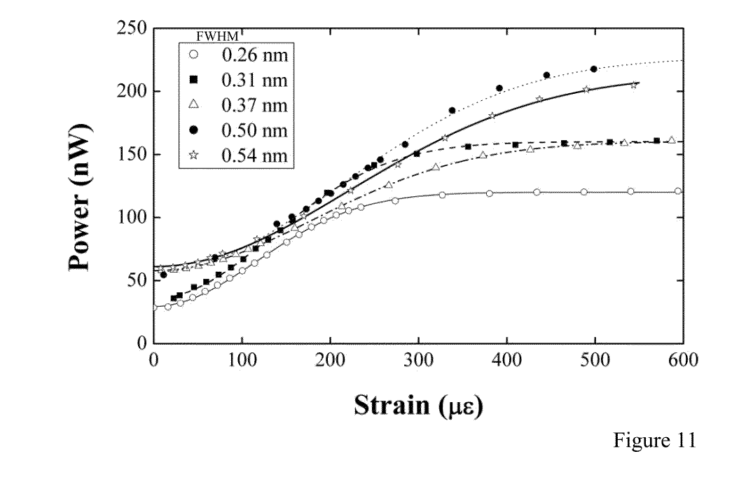

The results of strain calibrations for the different pairs of gratings tested are shown in FIG. 11. The data have been fitted with the theoretical curves given by Equation (4). The fitting parameters allowed any initial wavelength mismatch between the individual gratings used in a pair to be corrected for, and this has been taken into account in the data shown in FIG. 11. As can be seen there is good correlation between the theoretical fits (from the analytical model) and the experimental data (all fits had r²>0.99). The good match between theory and experimental data justifies the assumption that the grating spectral characteristics can be approximated by a Gaussian distribution in the theory, i.e., ignoring sidelobes and other secondary features.

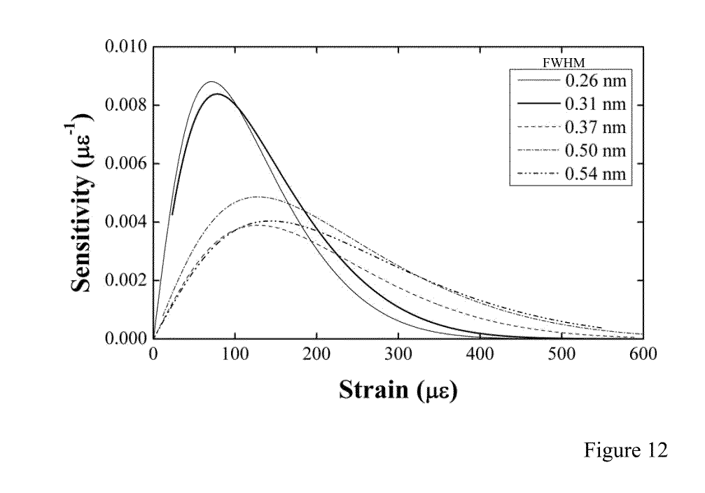

The relative sensitivity of the results shown in FIG. 11 to changes in applied strain was estimated using the fitted curves and is shown in FIG. 12. The results have been corrected for initial wavelength mismatches between the paired gratings and in general follow the trend predicted from theory (see FIG. 7). According to Equation (6) the absolute magnitude of the sensitivity depends on several of the grating parameters, including the magnitude of the reflectance of the reference grating (i.e., A₂). These factors, which have not been accounted for in FIG. 12, are believed to be the source of the differences observed between the theory and the experimental data.

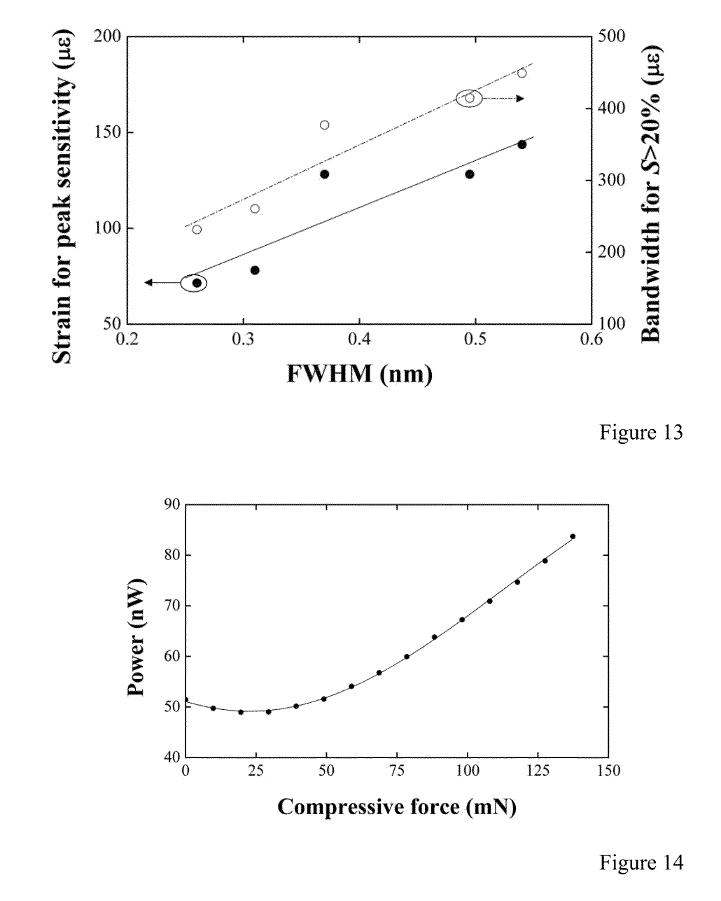

The strains at which the peak sensitivity occurs and the strain ranges over which the sensitivity is greater than 20% of the peak sensitivity for the experimentally determined data are shown in FIG. 13. The increasing trend of both these values with increased FWHM follows the trend predicted by the theory (see FIG. 8).

FIG. 14 shows the change in measured signal for a compression calibration carried out with the matched FBG system of FIG. 9. The length of compressed fiber used in this test was ˜10 mm. For a 125 μm diameter fiber of this length and the test configuration used, the critical buckling force was assumed to be about 180 mN. The Fbg4 grating pair (FWHM ˜0.5 nm) was used. As expected the measured power increases as the compressive force increases. The initial small drop in power as the compressive force increases is due to a slight mismatch between the centre wavelengths of the grating pair at zero force.

The integrated power measured by the OSA was normalized against the power measured by the second reference detector. This provides compensation for fluctuations in the intensity of light from the light source 12 and other non-measurand induced fluctuations in the light intensity.

The preceding theoretical analysis and experimental results demonstrate that the interrogator 9 may be programmed with data and algorithms that either define a mathematical relationship between wavelength shift in measured reflections from the FBG 8, or which define measurement values for particular detected wavelength shifts, for example in a look-up table. Those skilled in the relevant arts will appreciate that alternative mathematical models and variations in data forming look-up tables and the like are possible and will be of utility in a sensor for medical implants and that such arrangements are intended to fall within the scope of the disclosure of the invention.

It will be understood that the invention disclosed and defined in this specification extends to all alternative combinations of two or more of the individual features mentioned or evident from the text or drawings. All of these different combinations constitute various alternative aspects of the invention.

Need statement to change to an advantage—Ideally the surgeon is provided with real time feedback of their actions, for example to help ensure that the electrode assembly is being properly inserted. Conventional electrical sensors are not appropriate in this environment due to their size and the potential for interference. In contrast, optical fiber sensors have compact dimensions and immunity to electromagnetic interference. Significantly, an optical fiber sensor will not affect the current levels of compatibility of the implant with MRI scans.

What is claimed is:

1. An implantable medical device comprising:
a carrier member configured for implantation into a patient, the carrier member having a patient contact region;
one or more operative components disposed in the carrier member;
a fiber optic sensor including a fiber Bragg grating (FBG) disposed in the patient contact region of the carrier member; and, and an optical fiber extending from the FBG.

2. The device of claim 1, wherein the carrier member has a first elastic modulus and the fiber optic sensor has a second elastic modulus and wherein the first elastic modulus is less than the second elastic modulus.

3. The device of claim 2, wherein the first elastic modulus is in the range of approximately 1 to approximately 100 GPa and the second elastic modulus is less than or equal to approximately 100 MPa.

4. The device of claim 2, wherein:
the carrier member is elongate and has a distal tip region that is inserted into a cochlea during implantation, wherein the tip region physically contacts the patient during implantation;
the FBG is located at said tip region.

5. The device of claim 1, wherein the FBG is between approximately 0.3-01-0.5 mm in length.

6. An interrogator for a sensor fiber Bragg grating, the interrogator comprising:
a light source for providing incident light to the sensor fiber Bragg grating over a forward light path;
a return light path for receiving return light from the sensor fiber Bragg grating;
a reference fiber Bragg grating disposed in the return light path, the reference fiber Bragg grating having a grating pattern to reflect return light received from the sensor fiber Bragg grating, whereby variations in the environment of the sensor fiber Bragg grating within an operating range of the sensor fiber Bragg grating that affect the return light, result in variations in the proportion of the return light passed by the reference fiber Bragg grating; and
a light detector for receiving and detecting the return light passed by the reference fiber Bragg grating.

7. The interrogator of claim 6, wherein the return light path comprises a first light path in which the reference fiber Bragg grating is located, and a second light path, which does not include a said reference fiber Bragg grating, and wherein the light detector is adapted to also receive return light over the second light path and use the return light of the second light path as a compensation signal.

8. The interrogator of claim 7, wherein the light detector uses the return light of the second light path to compensate for at least one of fluctuations in the intensity of light generated by the light source and fluctuations in the intensity of the incident light.

9. The interrogator of claim 6, wherein for at least one environmental condition within the operating range of the sensor fiber Bragg grating, the return light passed by the reference fiber grating has two components centred at different wavelengths and wherein the interrogator comprises an additional fiber Bragg grating that reflects more of one of the components than the other.

10. A method of implanting a medical implant for providing stimulation signals to the nervous system of a recipient, the method comprising:
using a medical implant comprising a flexible carrier at a leading end of the medical implant as it is implanted in the recipient, the flexible carrier carrying a fiber optic including a fiber Bragg grating;
connecting the fiber optic to an interrogator, including a light detector for detecting variations in the reflection/transmission characteristics of the fiber Bragg grating responsive to forces applied to the fiber Bragg grating through the flexible carrier and provide an output indicative of the force applied to the fiber Bragg grating; and
as the medical implant is implanted in the recipient, monitoring the outputx.

11. The method of claim 10, wherein the medical implant is a cochlear implant including an array of electrode contacts carried by the flexible carrier.
